(19) United States
(12) Patent Application Publication
(10) Pub. No.: US 2010/0305060 A1
(43) Pub. Date: Dec. 2, 2010

(54) Nucleoside Prodrugs and Uses Thereof

(75) Inventors: Scott J. Hecker, Del Mar, CA (US); K. Raja Reddy, San Diego, CA (US)

Correspondence Address: SALIWANCHIK LLOYD & SALIWANCHIK;A PROFESSIONAL ASSOCIATION, PO Box 142950, GAINESVILLE, FL, 32614, US

(73) Assignee: Ligand Pharmaceuticals Incorporated, La Jolla, CA (US)

(21) Appl. No.: 12/745,419

(22) PCT Filed: Nov. 26, 2008
(86) PCT No.: PCT/US08/84828

§ 371 Date: May 28, 2010

Related U.S. Application Data
(60) Provisional application No. 60/991,163, filed on Nov. 29, 2007.

(51) Int. Cl. A61K 31/7072; C07H 19/11; A61P 31/14; A61P 31/12

(52) U.S. Cl. 514/51; 536/26.14

(57) ABSTRACT

This application provides compositions or compounds as disclosed herein that are suitable for treating hepatic viral diseases, such as hepatitis C(HCV).

### BACKGROUND OF THE INVENTION

Hepatitis C virus NS5B polymerase is an RNA-dependent RNA polymerase (RdRp), an enzyme that replicates RNA using an RNA template. In recent years there has been much interest in identifying nucleoside inhibitors of the NS5B polymerase for treatment of hepatitis C virus (HCV) infection. While initial reports from researchers in this area described modest efficacy, the tremendous potential of this class of agents became clear when Merck presented the results of evaluation of MK-0608 in the chimpanzee [Olsen, D. B.; Carroll, S. S.; Davies, M. E.; Handt, L.; Koeplinger, K.; Zhang, R.; Ludmerer, S.; MacCoss, M.; Hazuda, D. J. Robust suppression of viral replication in HCV infected chimpanzees by a nucleoside inhibitor of the NS5B polymerase. Antivir. Ther. 2006, 11 (5, Suppl.): S7. (15th International HIV Drug Resistance Workshop, Jun. 13-17, 2006; Sitges, Spain.]. In this study, a >5 log₁₀ reduction in viral titre was achieved in 7 days at an intravenous dose of 2 mg/kg/day.

Other agents advanced into development have not achieved such dramatic efficacy. For example, NM283 (Idenix, recently discontinued) achieved only a 1.15 log₁₀ reduction in viral titre in the chimpanzee (7 days, 16.6 mg/kg/day). [Standring D. N.; Lanford R.; Wright T.; Chung R. T.; Bichko V.; Cretton-Scott E.; Pan-Zhou X.; Bergelson S.; Qu L.; Tausek M.; Bridges E.; Moussa A.; Storer R.; Pierra C.; Benzaria S.; Gosselin G.; La Colla P.; Sommadossi J.P. NM 283 has potent antiviral activity against genotype 1 chronic hepatitis C virus (HCV-1) infection in the chimpanzee. J. Hepatology, 2003, 38, (Supp 2), 3.] In a phase 2 study, 81626 (Roche) at 1500 mg BID for 14 days achieved a mean reduction in serum viral titre of 1.2 log₁₀. [Roberts, S.; Cooksley, G.; Shaw, D.; Berns, H. K.; Brandt, M. T.; Feltner, S. H.; Hill, G.; Ipe, D.; Klumpp, K.; Mannino, M.; O'Mara, E.; Tu, Y.; Washington, C. B. Interim results of a multiple ascending dose study of R1626, a novel nucleoside analog targeting HCV polymerase in chronic HCV patients. 41^{st} Annual Meeting of the European Association for the Study of the Liver, Vienna, Apr. 26-30, 2006.]

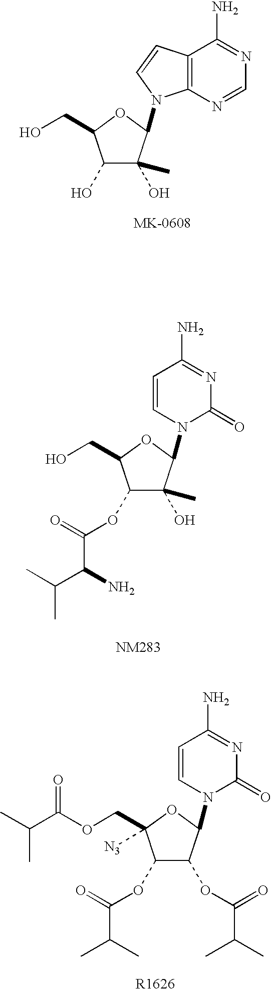

The difference in efficacy between these agents, all of which display good plasma exposure to the nucleoside, is likely due to differences in rates of phosphorylation and resulting levels of triphosphate in the liver. Conversion of nucleoside analogs to the active triphosphate form (NTP) requires the successive action of three kinase enzymes. These enzymes are sensitive to structural changes in their substrates, and consequently discovery programs seeking novel nucleosides often encounter inefficient conversion to the NTP. In many cases it is the action of the first kinase, which converts the nucleoside to its monophosphate (NMP), that is most greatly affected. A well-recognized method of circumventing a slow rate of initial nucleoside phorphorylation is to utilize a prodrug of the nucleoside monophosphate; this approach is termed “kinase bypass.” A number of prodrugs have been explored for this purpose, although few have been shown to achieve oral bioavailability and intracellular delivery of the monophosphate in vivo.

One such class of prodrugs is the aryl amidate (McGuigan) type. While these prodrugs have shown impressive intracellular delivery of monophosphates in vitro, there are few reports of in vivo application. (By contrast, their track record with phosphonates has been better.) One notable report by McGuigan details pharmacokinetic evaluation in the cynomolgus monkey of an aryl amidate prodrug of abacavir. [McGuigan, C.; Harris, S. A.; Daluge, S. M.; Gudmundsson, K. S.; McLean, E. W.; Burnette, T. C.; Marr, H.; Hazen, R.; Condreay, L. D.; Johnson, L.; De Clercq, E.; Balzarini, J. Application of phosphoramidate pronucleotide technology to abacavir leads to a significant enhancement of antiviral potency. [J. Med. Chem. 2005, 48, 3504-3515.] This article reports extremely rapid clearance of the prodrug from plasma when administered i.v. The dearth of other literature reports of in vivo characterization of aryl amidate prodrugs, despite numerous applications citing in vitro characterization, suggests that these prodrugs are not sufficiently stable for successful in vivo application. Moreover, one of the byproducts, typically a phenol, may be associated with potential toxicologic risks, especially if the human dose is high.

An alternative class of monophosphate prodrugs, showing excellent stability as well as the ability to target the liver, are the cyclic 1-aryl-1,3-propanyl ester (HepDirect) prodrugs. [Erion, M. D.; Reddy, K. R.; Boyer, S. H.; Matelich, M. C.; Gomez-Galeno, J.; Lemus, R. H.; Ugarkar, B. G.; Colby, T. J.; Schanzer, J.; Van Poelje, P. D. Design, synthesis, and characterization of a series of cytochrome P(450) 3A-activated prodrugs (HepDirect prodrugs) useful for targeting phosph(on)ate-based drugs to the liver. J. Am. Chem. Soc. 2004, 126, 5154-5163; Erion, M. D.; van Poelje, P. D.; Mackenna, D. A.; Colby, T. J.; Montag, A. C.; Fujitaki, J. M.; Linemeyer, D. L.; Bullough, D. A. Liver-targeted drug delivery using HepDirect prodrugs. J. Pharmacol. Exp. Ther. 2005, 312, 554-560.] Achievement of intracellular delivery of the nucleoside monophosphate in vivo has been demonstrated with MB07133, a prodrug of the monophosphate of the nucleoside cytarabine (araC), which has been advanced to human clinical trials for the treatment of hepatocellular carincoma. [Boyer, S. H.; Sun, Z.; Jiang, H.; Esterbrook, J.; Gomez-Galeno, J. E.; Craigo, W.; Reddy, K. R.; Ugarkar, B. G.; MacKenna, D. A.; Erion, M. D. Synthesis and characterization of a novel liver-targeted prodrug of cytosine-1-b-D-arabinofuranoside monophosphate for the treatment of hepatocellular carcinoma. J. Med. Chem. 2006, 49, 7711-7720.]

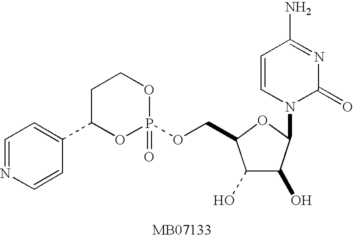

It is not possible to predict the utility of applying the kinase bypass approach to a previously unexplored nucleoside analog that is poorly converted to the triphosphate form in cells. For example, even if the prodrug moiety is cleaved at a sufficient rate, conversion to the triphosphate form requires the successive action of two different kinases and it is unknown whether the monophosphate and diphosphate intermediates will be substrates for these kinases.

### DESCRIPTION OF THE INVENTION

Work has recently been published surrounding the RdRp inhibitor 2′-fluoro-2-methylcytidine (PSI-6130) and its uridine analog RO2433. [Ma, H.; Jiang, W.-R.; Robledo, N.; Leveque, V.; Ali, S.; Lara-Jaime, T.; Masjedizadeh, M.; Smith, D. B.; Cammack, N.; Klumpp, K.; Symons, J. Characterization of the metabolic activation of hepatitis C virus nucleoside inhibitor β-D-2′-deoxy-2′-fluoro-2′-C-methylcytidine (PSI-6130) and identification of a novel active 5′-triphosphate species. J. Biol. Chem. 2007, 282, 29812-29820; Murkami, E.; Niu, C.; Bao, H.; Steuer, H. M. M.; Whitaker, T.; Nachman, T.; Sofia, M. A.; Wang, P.; Otto, M. J.; Furman, P. A. The Mechanism of Action of β-D-2′-C-Methylcytidine Involves a Second Metabolic Pathway Leading to β-D-2-C-Methyluridine 5′-Triphosphate, a Potent Inhibitor of the Hepatitis C Virus RNA-Dependent RNA Polymerase. Antimicrob. Agents Chemotherapy, 2008, 52, 458-464.] Both compounds as their triphosphates are potent at the enzyme level (IC₅₀s of 0.13 and 0.52 μM, respectively); however, PSI-6130 is potent in the replicon assay, whereas RO2433 is inactive (EC₅₀>100 μM). Restoration of replicon activity through use of an aryl amidate prodrug of the monophosphate (PSI-7672) was demonstrated. More recently, Pharmasset reported the nomination for development of PSI-7851 (structure undisclosed), an aryl amidate prodrug of RO2433 (Pharmasset, Inc. Press Release, May 19, 2008).

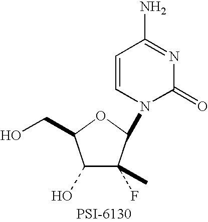

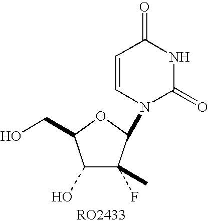

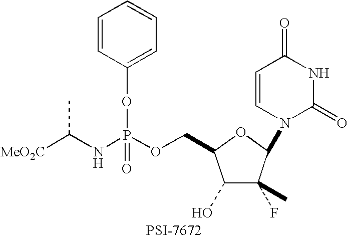

| | NS5b IC₅₀ of NTP (μM) | Replicon EC₅₀ (μM) |
|---|---|---|
| PSI-6130 | 0.13 | 0.6 |
| RO2433 | 0.52 | >100 |
| PSI-7672 | — | 1.6 |

To explore the utility of HepDirect prodrugs and a kinase bypass for PSI-6130 and RO2433, the two compounds below were synthesized. Compound A is a HepDirect prodrug of the 5′-monophosphate of PSI-6130. Compound B is a HepDirect prodrug of the 5′-monophosphate of RO2433, and additionally contains a 3′-O-propionyl group to aid oral absorption.

Ester prodrugs of hydroxyl groups can remove a hydrogen-bond donor while increasing lipophilicity, thereby increasing the rate of permeation of a compound across intestinal epithelial cells. It is expected that this ester group is cleaved by esterase enzymes upon entering the systemic circulation. In addition to simple acyl groups (such as C₁- to C₆-acyl), it is envisioned that esters derived from acylation with a wide variety of carboxylic acids will impart the desired properties of enhancing oral bioavailability while being cleaved by esterase enzymes following absorption. For example, the acyl group can contain ether oxygen atoms, or can contain a 5- or 6-membered ring heterocycle (i.e., the acyl group may be derived from furancarboxylic acid, pyrazolecarboxylic acid, oxazolecarboxylic acid, or the like.) In addition, simple alkyl carbonate derivatives can achieve the desired properties.

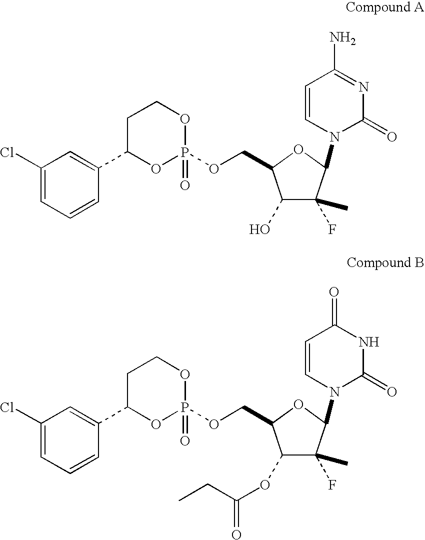

The two nucleosides and their respective prodrugs, Compounds A and B, were evaluated for production of nucleoside triphosphate in vitro in rat hepatocytes and in vivo in rat livers following intraperitoneal and oral dosing. As shown in the table below, application of the HepDirect prodrug technology to PSI-6130 (resulting in Compound A) achieves only a slight increase in triphosphate levels, both in vitro and in vivo. On the other hand, application to RO2433 (resulting in Compound B), affords a dramatic increase in triphosphate levels. In hepatocytes, Compound B results in an NTP concentration of 87.8 nmol/g. In rats, an NTP concentration of 78.4 nmol/g is observed 3 hours following an intraperitoneal dose of 5 mg/kg (nucleoside equivalents, n.e.), and a concentration of 26.5 nmol/g is observed 5 hours following an oral dose of 10 mg/kg (n.e.). In each case, nmol/g refers to nanomoles per gram of hepatocytes or harvested rat liver tissue (i.e., per unit wet mass).

| | | Rat liver | | Rat liver | |
|---|---|---|---|---|---|
| | Rat hepatocyte | [NTP] @ | | [NTP] @ | |
| | [NTP] @ | t (5 mg/kg* | | t (10 mg/kg* | |
| | 2 h (10 μM, | i.p., nmol/g) | | p.o., nmol/g) | |
| | nmol/g) | 3 hr | 5 hr | 3 hr | 5 hr |
| PSI-6130 | <1.0 | <0.04 | <0.04 | — | — |
| Compound A | 1.0 | 0.33 | 0.05 | — | |
| RO2433 | <2.50 | 0.73 | 3.59 | 1.71 | 1.17 |
| Compound B | 87.8 | 78.4 | 58.0 | 23.3 | 26.5 |

*nucleoside equivalents

By comparison, PSI-7851 (an aryl amidate prodrug of the monophosphate of RO2433; exact structure not disclosed) was recently reported to achieve liver triphosphate levels of 2550 ng/g with a 50 mg/kg oral dose in the rat [Furman, P. A.; Wang, P.; Niu, C.; Bao, D.; Symonds, W.; Nagarathnam, D.; Micolochick Steuer, H.; Rachakonda, S.; Ross, B. S.; Otto, M. J.; Sofia, M. PSI-7851: A Novel Liver-Targeting Nucleotide Prodrug for the Treatment of Hepatitis C. P. A. Furman; P. Wang; C. Niu; D. Bao; W. Symonds; D. Nagarathnam; H. Micolochick Steuer; S. Rachakonda; B. S. Ross; M. J. Otto; M. Sofia. Abstracts of the 2008 Meeting of the American Association for the Study of Liver Diseases; Abstract #1901]. Assuming a molecular weight of 500 for PSI-7851, this level corresponds to 5.1 nmol/g. Based on these results, it is estimated that Compound B, given at an equimolar dose, would deliver >10-fold higher levels of triphosphate to the liver.

It is envisioned that certain moieties can serve as prodrugs for the uridine portion of RO2433, which can be useful for tuning lipophilicity as well as masking the hydrogen-bond donor functionality. Simple O-acyl derivatives are one such type of prodrug. In addition, it is known that the monophosphate of PSI-6130 is deaminated within cells to afford the monophosphate of RO2433 [Ma, H.; Jiang, W.-R.; Robledo, N.; Leveque, V.; Ali, S.; Lara-Jaime, T.; Masjedizadeh, M.; Smith, D. B.; Cammack, N.; Klumpp, K.; Symons, J. Characterization of the metabolic activation of hepatitis C virus nucleoside inhibitor [3-D-2′-deoxy-2′-fluoro-2′-C-methylcytidine (PSI-6130) and identification of a novel active 5′-triphosphate species. J. Biol. Chem. 2007, 282, 29812-29820.] Therefore, 4-O-alkyl and 4-N-alkyl derivatives can also be deaminated, and thereby can serve as prodrugs for the uridine moiety.

### DEFINITIONS

The term “acyl” refers to RC(O)— wherein R is an aryl or aliphatic group.

The term “aliphatic” refers to a hydrocarbon group that is a straight chain, a branched chain, a ring, or any combination thereof. Aliphatic groups may be saturated or unsaturated, but are not aromatic. Unsaturated aliphatic groups contain one or more double or triple bonds.

The term “alkoxy” refers to R—O— wherein R is an alkyl group. As used within this application, C₁-C₆ alkoxy provides support for R groups that can be: a methyl group, C₁-C₂ alkyl groups, C₁-C₃ alkyl groups, C₁-C₄ alkyl groups, or C₁-C₅ alkyl groups.

The term “alkyl” refers to a saturated hydrocarbon group that is a straight chain, a branched chain, a ring, or any combination thereof. As used within this application, C₁-C₆ alkyl provides support for: a methyl group, C₁-C₂ alkyl groups, C₁-C₃ alkyl groups, C₁-C₄ alkyl groups, or C₁-C₅ alkyl groups.

The term “aromatic” refers to a group containing at least one aromatic ring.

The term “aryl” refers to an aromatic hydrocarbon group. As used herein, unsubstituted aryl refers to an aromatic group consisting of hydrogen and carbon wherein each carbon is a ring atom. Aryl groups, as defined herein, will typically have six carbon atoms; thus, phenyl is an example of a suitable unsubstituted aryl group and ortho-tolyl is an example of a suitable substituted aryl group.

The term “carbocyclic” refers to a ring structure in which every ring atom is carbon. Phenyl and cyclopentyl are non-limiting examples of carbocyclic groups. Carbocyclic structures can have 3, 4, 5, 6, 7, 8, 9, 10, 11, 12, 13, 14, 15, 16, 17, 18, 19 or 20 ring atoms.

The term “cyano” refers to the group containing a single nitrogen triple-bonded to a single carbon.

The term “halogen” refers to —F, —Cl, —Br, or —I.

The term “heteroaryl” refers to a heterocyclic aromatic group. As used herein, unsubstituted heteroaryl refers to a heterocyclic aromatic group wherein each non-hydrogen atom is a ring atom. Heteroaryl groups, as defined herein, will typically have at least five ring atoms (e.g., five or six ring atoms of which 1, 2 or 3 ring atoms are an atom other than carbon (e.g., O, S or N). Thiazolyl and pyridyl are non-limiting examples of unsubstituted heteroaryl groups.

The term “heterocyclic” refers to a ring structure in which at least one ring atom is carbon and at least one ring atom is an atom other than carbon (such as O, S, or N). A heterocyclic group can be aromatic or non-aromatic. Piperadine and oxetane are non-limiting examples of non-aromatic heterocycles. Thiazole and pyridine are non-limiting examples of aromatic heterocycles. Heterocyclic ring structures can have 3, 4, 5, 6, 7, 8, 9, 10, 11, 12, 13, 14, 15, 16, 17, 18, 19 or 20 ring atoms, of which at least 1, 2 or 3 ring atoms are an atom other than carbon (such as O, S, or N).

The term “hydrocarbon” refers to a group containing carbon and hydrogen atoms only. Groups containing hydrocarbons, such as aliphatic groups or alkyl groups, can have 1, 2, 3, 4, 5, 6, 7, 8, 9, 10, 11, 12, 13, 14, 15, 16, 17, 18, 19 or 20 carbon atoms.

The term “lower” refers to groups having between one and six atoms.

The term “pharmaceutically acceptable salts” refers to salts prepared from pharmaceutically acceptable non-toxic bases or acids including inorganic or organic bases and inorganic or organic acids. Where the non-salt form of the compound has multiple acidic or basic functional groups, one or two or three or more of the functional groups may be converted to the salt form. Inorganic salts include aluminum, ammonium, calcium, copper, ferric, ferrous, lithium, magnesium, manganic, manganous, potassium, sodium, zinc, and the like. Particularly preferred are the ammonium, calcium, magnesium, potassium, and sodium salts. Salts in the solid form may exist in the form of hydrates or other pure or mixed solvates of one or more non-toxic solvents. Salts in the solid form may exist in more than one crystal structure. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylene-diamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. When the compound of the present invention is basic, salts may be prepared from pharmaceutically acceptable non-toxic acids, including inorganic and organic acids. Such acids include acetic, trifluoroacetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are citric, hydrobromic, hydrochloric, trifluoroacetic, maleic, phosphoric, sulfuric, fumaric, and tartaric acids.

### Compounds and Uses Thereof

One aspect of the present invention provides compounds of general formula III

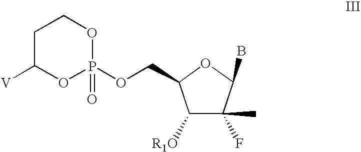

wherein V is selected from the group consisting of optionally substituted (e.g., unsubstituted or substituted by one or two groups selected from halogen, trifluoromethyl, C₁-C₆ (lower) alkyl, C₁-C₆ (lower) alkoxy, and cyano) monocyclic aryl (e.g., phenyl) and optionally substituted (e.g., unsubstituted or substituted by one or two groups selected from halogen, trifluoromethyl, C₁-C₆ (lower) alkyl, C₁-C₆ (lower) alkoxy, and cyano) monocyclic heteroaryl,

### B is

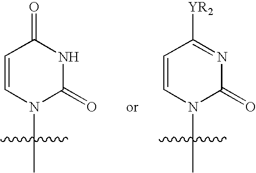

R₁ is selected from H and COR₃;

### Y is O or NH;

R₂ is C₁-C₆ alkyl or COR₃, and
R₃ is C₁₋₆ alkyl, C₁₋₃ alkoxy-C₁₋₆ alkyl, C₃₋₆ cycloalkyl, C₃₋₆ cycloalkyl-C₁₋₃ alkyl, C₁₋₆ alkoxy, or heteroaryl wherein the heteroaryl is a five-membered ring containing one or two heteroatoms selected from nitrogen, oxygen or sulfur, or the heteroaryl is a six-membered ring containing one or two nitrogen atoms. In one embodiment, R₂ is C₁-C₆ alkyl. In various aspects of the invention, R₁ and/or R₂ can be COR₃. The phrase “R₁ and/or R₂ are COR₃” is used to indicate that each of R₁ and R₂ can, independently, be COR₃ when both R₁ and R₂ are present in the compound described herein. Thus, R₁ alone can be COR₃ (R₂ is a C₁-C₆ alkyl group), R₂ alone can be COR₃ (R₁ is H) or both R₁ and R₂ are COR₃. Accordingly, various embodiments are provided where: a) R₁ and/or R₂ is COR₃ and R₃ is C₁-C₆ alkyl; b) R₁ and/or R₂ is COR₃ and R₃ is C₁₋₃ alkoxy-C₁₋₆ alkyl; c) R₁ and/or R₂ is COR₃ and R₃ is C₃₋₆ cycloalkyl; d) R₁ and/or R₂ is COR₃ and R₃ is C₃₋₆ cycloalkyl-C₁₋₃ alkyl; e) R₁ and/or R₂ is COR₃ and R₃ is C₁₋₆ alkoxy; 0 R₁ and/or R₂ is COR₃ and R₃ is a heteroaryl that is a five-membered ring containing one or two heteroatoms selected from nitrogen, oxygen or sulfur; or g), R₁ and/or R₂ is COR₃ and R₃ is a heteroaryl that is a six-membered ring containing one or two nitrogen atoms.

In various additional aspects of the invention, compositions comprising pharmaceutically acceptable excipients, carriers, stabilizers or diluents and the compounds of the invention are also provided. The subject application also provides methods of treating hepatic viral diseases, such as hepatitis C(HCV), comprising the administration of compositions or compounds as provided herein to an individual in need of such treatment.

Any accepted mode of administration can be used and determined by those skilled in the art. For example, administration may be by various parenteral routes such as subcutaneous, intravenous, intradermal, intramuscular, intraperitoneal, intranasal, transdermal, oral, or buccal routes. Parenteral administration can be by bolus injection or by gradual perfusion over time. Preparations for parenteral administration include sterile aqueous or non-aqueous solutions, suspensions, and emulsions, which may contain auxiliary agents or excipients which are known in the art, and can be prepared according to routine methods. Aqueous injection suspensions that may contain substances which increase the viscosity of the suspension include, for example, sodium carboxymethyl cellulose, sorbitol, and/or dextran. Optionally, the suspension may also contain stabilizers. Pharmaceutical compositions include suitable solutions for administration by injection, and contain from about 0.01 to 99 percent, preferably from about 20 to 75 percent of active compound together with the excipient.

### Methods of Synthesis

The schemes below depict methods by which the compounds described herein can be prepared.

Prodrugs of 2′-deoxy-2′-fluoro-2′-C-methyl-cytidine monophosphate The nucleoside 2′-deoxy-2′-fluoro-2′-C-methyl-cytidine (1) was prepared as described in J Med. Chem. 2005, 48, 5504-5508. Selective protection of the 3′-hydroxyl group is achieved by bis-3′,5′-O-silylation followed by selective deprotection at 5′ with aqueous trifluoroacetic acid. Phosphorylation of the 5′-hydroxyl group to give 4, using reagents such as 3, is achieved using reported methods (for example, see Hecker, S. J.; Reddy, K. R.; Van Poelje, P. D.; Sun, Z.; Huang, W.; Varkhedkar, V.; Reddy, M. V.; Fujitaki, J. M.; Olsen, D. B.; Koeplinger, K. A.; Boyer, S. H.; Linemeyer, D. L.; MacCoss, M.; Erion, M. D. Liver-targeted prodrugs of 2′-methyladenosine for therapy of hepatitis C virus infection. J. Med. Chem. 2007, 50, 3891-3896, and references therein). The 3′-O-t-butyldimethylsilyl protecting group is removed with tetraethylammonium fluoride, affording HepDirect prodrug 5 (Compound A).

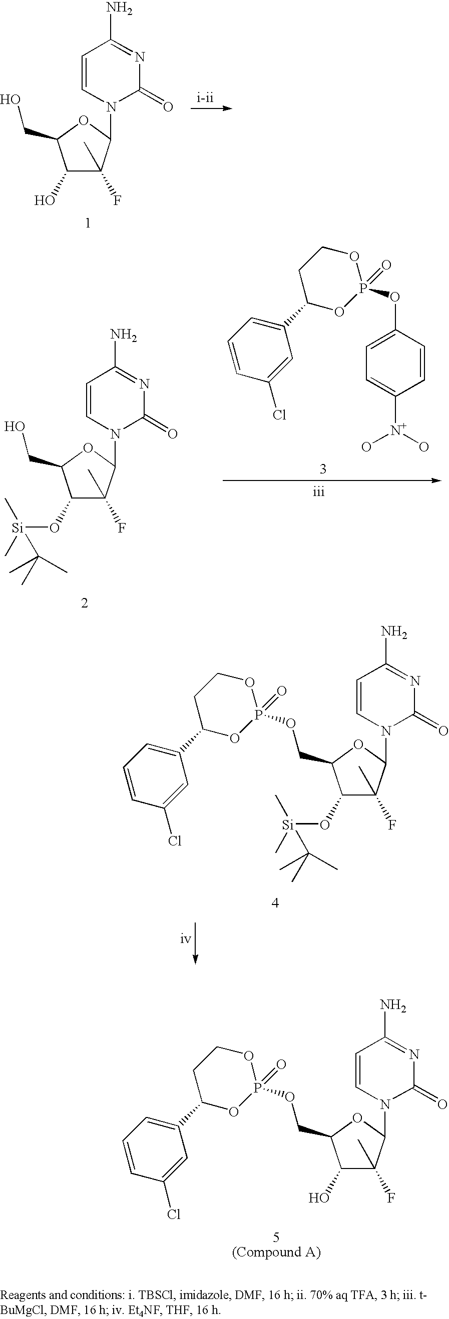

Prodrugs of 2′-deoxy-2′-fluoro-2′-C-methyl-uridine monophosphate

The nucleoside 2′-deoxy-2′-fluoro-2′-C-methyl-uridine (6) was prepared as described in J. Med. Chem. 2005, 48, 5504-5508. Using methods similar to those described above for the cytidine analog, one may prepare analogous prodrugs in the uridine series. Alternatively, with a simple modification, one may prepare compounds containing a 3′-O-acyl group. For example, selective 5′-O-silylation followed by acylation at 3′ affords intermediate 7, which is desilylated to give compound 8. Phosphorylation with reagents such as 3 affords the desired 3′-O-acyl HepDirect prodrugs such as 9 (Compound B).

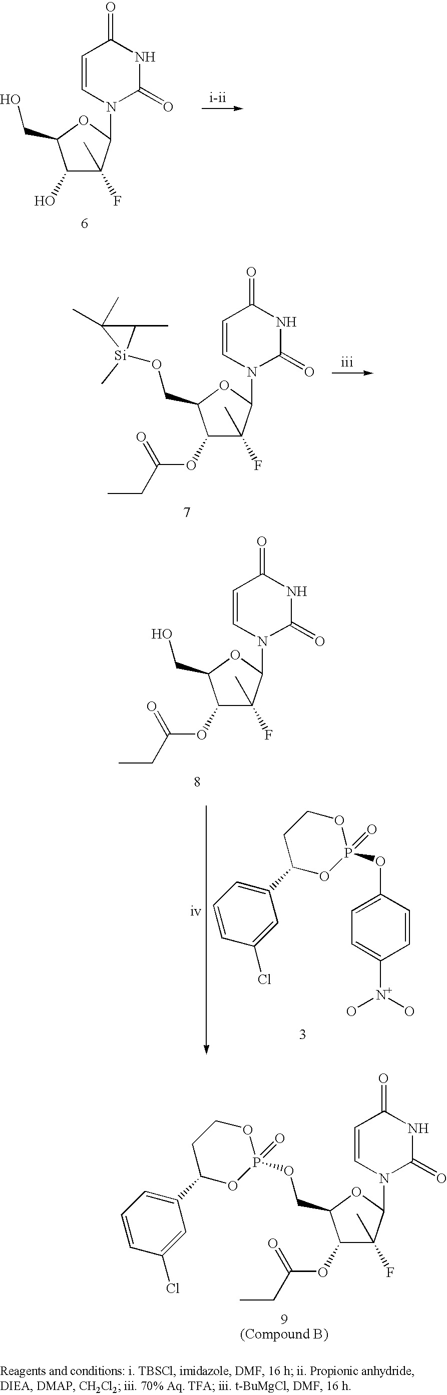

### EXAMPLES

### Example 1

### Preparation of 2 ′-deoxy-2 ′-fluoro-2′-C-methyl-cis-5′-O—[4(S)-(3-chlorophenyl)-2-oxo-1,3,2-dioxaphosphorinan-2-yl]1 cytidine (Compound A)

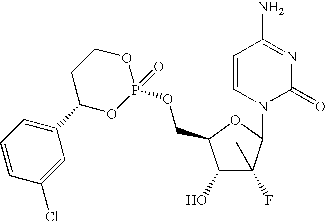

2′-Deoxy-2′-fluoro-2′-C-methyl-cytidine was prepared as described in J. Med. Chem. 2005, 48, 5504-5508.

### Step A:

### Preparation of 3′,5′-di(tert-butyldimethylsilyl)-2′-deoxy-2′-fluoro-2′-C-methyl-cytidine

To a solution of 2′-deoxy-2′-fluoro-2′-C-methyl-cytidine (1 mmol) in DMF (5 mL) was added imidazole (4 mmol) followed by tert-butylchlorodimethylsilane (2 mmol) at room temperature. The reaction was stirred at room temperature overnight and concentrated under reduced pressure. The concentrate was diluted with ethyl acetate (100 mL), washed with aq ammonium chloride (2×10 mL) followed by water (2×10 mL) and dried. The crude was chromatographed to get pure 3′,5′-di(tert-butyldimethylsilyl)-2′-deoxy-2′-fluoro-2′-C-methyl-cytidine.

### Step B:

### Preparation of 3′-(tert-butyldimethylsilyl)-2′-deoxy-2′-fluoro-2′-C-methyl-cytidine

An ice-cold solution of 70% aq trifluoroacetic acid (4 mL) was added to 3′,5′-di(tert-butyldimethylsilyl)-2′-deoxy-2′-fluoro-2′-C-methyl-cytidine (1 mmol) at 0° C. The reaction mixture was stirred at 0° C. for 3 h and concentrated under reduced pressure at 0-5° C. The crude mixture was diluted with ethyl acetate (100 mL) and washed with sat aq sodium bicarbonate (2×10 mL), water (2×10 mL) and dried. The organic extract was concentrated and chromatographed to get pure 3′-(tert-butyldimethylsilyl)-2′-deoxy-2′-fluoro-2′-C-methyl-cytidine.

### Step C:

### Preparation of 3′-(tert-butyldimethylsilyl)-2′-deoxy-2′-fluoro-2′-C-methyl-cis-5′-β-[4(S)-(3-chlorophenyl)-2-oxo-1,3,2-dioxaphosphorinan-2-yl]cytidine

To a stirred solution of 3′-(tert-butyldimethylsilyl)-2′-deoxy-2′-fluoro-2′-C-methyl-cytidine (1 mmol) in DMF (5 mL) under a nitrogen atmosphere was added a solution of 1M t-butyl magnesium chloride in THF (2.4 mL, 2.4 mmol). The reaction was allowed to stir at ambient temperature for 30 min and the phosphorylating reagent (1.5 mmol) was added in one portion. The reaction mixture was stirred at room temperature for 16 h (TLC). The reaction was quenched with a saturated ammonium chloride solution and extracted with ethyl acetate (3×20 mL). The combined extracts were washed with water and dried. The solvent was removed under reduced pressure and the residue was purified by column chromatography to get 3′-(tert-butyldimethylsilyl)-2′-deoxy-2′-fluoro-2-C-methyl-cis-5′-O-[4 (S)-(3-chlorophenyl)-2-oxo-1,3,2-dioxaphosphorinan-2-yl]cytidine.

### Step D:

### Preparation of 2′-deoxy-2′-fluoro-2′-C-methyl-cis-5′-O[4(S)-(3-chlorophenyl)-2-oxo-1,3,2-dioxaphosphorinan-2-yl]cytidine

To a solution of 3′-(tert-butyldimethylsilyl)-2′-deoxy-2′-fluoro-2′-C-methyl-cis-5′-O—[4(S)-(3-chlorophenyl)-2-oxo-1,3,2-dioxaphosphorinan-2-yl]cytidine (1 mmol) in THF (10 mL) was added tetraethylammonium fluoride (1.5 mmol). The reaction was left stirring at room temperature for 16 h. The reaction mixture was concentrated upon complete conversion (TLC) and chromatographed to get pure 2′-deoxy-2′-fluoro-2′-C-methyl-cis-5′-O—[4(S)-(3-chlorophenyl)-2-oxo-1,3,2-dioxaphosphorinan-2-yl]cytidine. LC-MS calcd for C₁₉H₂₂ClFN₃O₇P [M+H]⁺, 489.8. found [M+H]⁺, 490.1. ¹H NMR (300 MHz, DMSO-d₆) δ: 1.16 (d, 3H, J=22 Hz), 2.10-2.25 (m, 2H), 3.55-3.85 (m, 1H), 3.90-4.10 (m, 1H), 4.20-4.60 (series of m, 4H), 5.60-5.78 (m, 21-1) (5.80-5.95 (m, 1H), 6.00-6.30 (m, 2H), 7.10-7.55 (m, 6H). ³¹P NMR (120 MHz, DMSO-d₆) δ: −6.30

### Example 2

### Preparation of 2′-deoxy-2′-fluoro-2′-C-methyl-3′-propionyl-cis-5′-O-14(S)-(3-chlorophenyl)-2-oxo-1,3,2-dioxaphosphorinan-2-yl]uridine (Compound B)

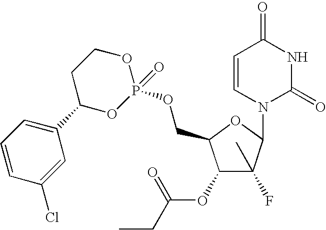

2′-Deoxy-2′-fluoro-2′-C-methyl-uridine was prepared as described in J. Med. Chem. 2005, 48, 5504-5508.

### Step A:

### Preparation of 5′-(tert-butyldimethylsilyl)-2′-deoxy-2′-fluoro-2′-C-methyl-uridine

To a solution of 2′-deoxy-2′-fluoro-2′-C-methyl-uridine (230 mg, 0.88 mmol) in DMF (10 mL) was added imidazole (144 mg, 2.12 mmol) followed by tert-butylchlorodimethylsilane (160 mg, 1.06 mmol) at room temperature. The reaction was stirred at room temperature overnight and concentrated under reduced pressure. The concentrate was diluted with ethyl acetate (100 mL) and washed with aq ammonium chloride (2×10 mL) followed by water (2×10 mL), brine (20 mL) and dried (Na₂SO₄). The crude was chromatographed with 0-15% dichloromethane-MeOH to get 160 mg (48%) of 5′-(tert-butyldimethylsilyl)-2′-deoxy-2′-fluoro-2′-C-methyl-uridine.

### Step B:

### Preparation of 5′-(tert-butyldimethylsilyl)-2′-deoxy-2′-fluoro-2′-C-methyl-3′-propionyl-uridine

To a solution of 5′-(tert-butyldimethylsilyl)-2′-deoxy-2′-fluoro-2′-C-methyl-uridine (160 mg, 0.428 mmol) in dichloromethane (20 mL) at 0° C. was added N,N-diisopropylethylamine (0.42 mL, 2.56 mmol) and 4-dimethylaminopyridine (12 mg, 0.09 mmol), followed by propionic anhydride (0.24 mL, 1.84 mmol). The reaction mixture was stirred at room temperature for 2 h, then diluted with aqueous saturated sodium bicarbonate (20 mL) and extracted with ethyl acetate (2×40 mL). The organic layer was washed with water (2×10 mL), brine (2×10 mL) and dried (Na₂SO₄). The crude obtained upon removal of solvent was chromatographed with 0-15% dichloromethane-MeOH to afford 180 mg (98%) of the title compound.

### Step C:

### Preparation of 2′-deoxy-2′-fluoro-2′-C-methyl-3′-propionyl-uridine

An ice-cold solution of 70% aq trifluoroacetic acid (4 mL) was added to 5′-(tert-butyldimethylsilyl)-2′-deoxy-2′-fluoro-2′-C-methyl-3′-propionyl-uridine (242 mg, 0.56 mmol) at 0° C. The reaction mixture was stirred at 0° C. for 3 h and concentrated under reduced pressure at 0-5° C. The concentrated reaction mixture was diluted with ethyl acetate (100 mL) and washed with sat aq sodium bicarbonate (2×10 mL), water (2×10 mL) and dried. The organic extract was concentrated and chromatographed by eluting with 0-15% dichloromethane-MeOH to get pure 2′-deoxy-2′-fluoro-2′-C-methyl-3′-propionyl-uridine (100 mg, 56%).

### Step D:

### Preparation of 2′-deoxy-2′-fluoro-2′-C-methyl-3′-propionyl-cis-5′-O[4(S)-(3-chlorophenyl)-2-oxo-1,3,2-dioxaphosphorinan-2-yl] uridine

To a stirred solution of 2′-deoxy-2′-fluoro-2′-C-methyl-3′-propionyl-uridine (50 mg, 0.15 mmol) in THF (2 mL) under nitrogen atmosphere was added a solution of 1M t-butyl magnesium chloride in THF (0.3 mL, 0.30 mmol). The reaction was allowed to stir at ambient temperature for 30 min and the phosphorylating reagent (87 mg, 0.23 mmol) was added in one portion. The reaction mixture was stirred at room temperature for 16 h (TLC). The reaction was quenched with a saturated ammonium chloride solution and extracted with ethyl acetate (3×20 mL). The combined extracts were washed with water and dried. The solvent was removed under reduced pressure and the residue was purified by column chromatography using 0-10% dichloromethane-MeOH to afford the pure phosphorylated product (40 mg, 51%). LC-MS calcd for C₂₂H₂₅ClFN₂O₉P [M+H]⁺546.8. found [M+H]⁺, 547.6. ¹H NMR (500 MHz, CD₃OD) δ: 1.14 (t, 3H, J=7 Hz), 1.38 (d, 3H, J=22 Hz), 2.20-2.30 (m, 1H), 2.38-2.50 (m, 3H) 4.30-4.75 (series of m, 5H), 5.22-5.40 (m, 1H), 5.58 (d, 1H, J=8.5 Hz), 5.70-5.76 (m, He, 6.05-6.20 (m, 1H), 7.30-7.45 (m, 3H), 7.50 (s, 1H), 7.63 (1H, d, J=8 Hz). ³¹P NMR (200 MHz, CD₃OD) 6: −4.53. Anal calcd for C₂₂H₂₅N₂O₉FC1P+0.3H₂O+0.4-CH₂Cl₂: C, 46.12; H, 4.55; N, 4.81. Found: C, 45.78; H, 4.08; N, 4.77.

### Example 3

### NTP Generation in Rat Hepatocytes

Hepatocytes were prepared from freely feeding male Sprague Dawley or Wistar (Han) rats (250-300g) according to the procedure of Berry and Friend (Berry, M. N., and D. S. Friend. High yield preparation of isolated rat liver parenchymal cells. J. Cell. Biol. 43: 506-520, 1969) as modified by Groen et al (Groen, A. K., H. J. Sips, R. C. Vervoon, and J. M. Tager. Intracellular compartmentation and control of alanine metabolism in rat liver parenchymal cells. Eur. J. Biochem. 122: 87-93, 1982). Hepatocytes (20 mg/mL wet weight, >85% trypan blue viability) were incubated at 37° C. in 2 mL of Krebs-bicarbonate buffer containing 20 mM glucose, and 1 mg/mL BSA for 2 h in the presence of 10 μM nucleoside or prodrug (from 10 mM stock solutions in DMSO). Following the incubation, 1600 μL aliquot of the cell suspension was centrifuged and 500 μL of 60% acetonitrile containing 0.1 mg/mL dicyclohexylcarbodiimide (DCCD) and 0.1% (v/v) ammonium hydroxide was added to the pellet and vigorously vortexed. After 10 min centrifugation at 14,000 rpm, the supernatant was analyzed by LC-MS/MS (Applied Biosystems, API 4000) equipped with an Agilent 1100 binary pump and a LEAP injector as described in Example 4. NTP was detected by using MS/MS mode (M₋/78.8) and quantified based on comparison to a standard of lamivudine 5′-triphosphate. Conversion of compounds to nucleoside triphosphate (NTP) in rat hepatocytes is shown in Table 1.

TABLE 1

| | Rat hepatocyte [NTP] |
|---|---|
| | @ 2 h (10 μM, nmol/g) |
| PSI-6130 | <1.0 |
| Compound A | 1.0 |
| RO2433 | <2.50 |
| Compound B | 87.8 |

### EXAMPLE 4

### Evaluation of Liver NTP Levels in Rat

Nucleoside analogues and their prodrugs were administered to fasted male Sprague-Dawley or Wistar (Han) rats by oral gavage. At 3 or 5 hours following drug administration, a lobe of the liver (−1 g) was freeze-clamped in liquid nitrogen and homogenized in 10 volumes of ice-cold 60% acetonitrile containing 1 mg/kg dicyclohexylcarbodiimide (DCCD) and 0.1% (v/v) ammonium hydroxide. Following centrifugation to clarify the homogenate, the supernatant was analyzed for nucleotides by an LC-MS/MS method as described below.

The extracts were analyzed by LC-MS/MS (Applied Biosystems, API 4000) equipped with an Agilent 1100 binary pump and a LEAP injector. Ten uL of sample was injected onto an Xterra MS C18 column (3.5 um, 2.1×50 mm, Waters Corp.) with a SecurityGuard C18 guard column (5 μm, 4.0×3.0 mm, Phenomenex) and eluted with a gradient mobile phase A and B (20 mM N,N-dimethylhexylamine and 10 mM propionic acid in 80% methanol) at a flow rate of 0.3 mL/min (0 min, 0% B, 0-1 min, 0-50% B; 1-3 min, 50-100% B, 3-6 min, 100% B; 6-6.1 min, 100-0% B; 6.1-9 min, 0% B). NTP was detected by using MS/MS mode (M778.8). The quantitative analysis of liver NTP was calculated based on a calibration curve generated using lamivudine 5′-triphosphate as the standard.

The concentration of nucleoside triphosphate in the liver 3 and 5 hours after an intraperitoneal and oral dose of the compounds is shown in Table 2.

TABLE 2

| | Rat liver [NTP] | | Rat liver [NTP] | |
|---|---|---|---|---|
| | (5 mg/kg* i.p., nmol/g) | | (10 mg/kg* p.o., nmol/g) | |
| | 3 hr | 5 hr | 3 hr | 5 hr |
| PSI-6130 | <0.04 | <0.04 | — | — |
| Compound A | 0.33 | 0.05 | — | — |
| RO2433 | 0.73 | 3.59 | 1.71 | 1.17 |
| Compound B | 78.4 | 58.0 | 23.3 | 26.5 |

*nucleoside equivalents

1-10. (canceled)

11. A compound of general formula III 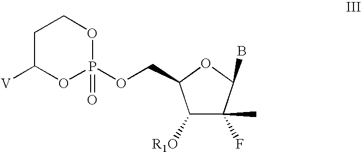
wherein
V is selected from the group consisting of phenyl and monocyclic heteroaryl, wherein (i) each said monocyclic heteroaryl contains five or six ring atoms of which 1 or 2 ring atoms are heteroatoms selected from the group consisting of N, S, and O, and the remainder of the ring atoms are carbon, and (ii) each said phenyl or monocyclic heteroaryl is unsubstituted or is substituted by one or two groups selected from halogen, trifluoromethyl, C₁-C₆ alkyl, C₁-C₆ alkoxy, and cyano;
B is 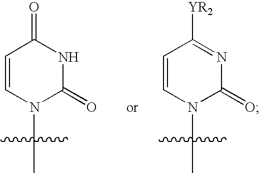
R₁ is selected from H and COR_{S};
Y is O or NH;
R₂ is C₁-C₆ alkyl or COR_{S}; and
R₃ is C₁-C₆ alkyl;
or a pharmaceutically acceptable salt thereof.

12. The compound of claim 11,
wherein B is 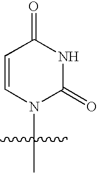
or a pharmaceutically acceptable salt thereof.

13. The compound of claim 11, wherein V is selected from the group consisting of phenyl and pyridyl, each unsubstituted or substituted by one or two halogens or a pharmaceutically acceptable salt thereof.

14. The compound of claim 11, wherein V is selected from the group consisting of 3-chlorophenyl, 3,5-dichlorophenyl, 3,5-difluorophenyl, 3-pyridyl and 4-pyridyl or a pharmaceutically acceptable salt thereof.

18. The compound of claim 11, wherein V is 3-chlorophenyl or a pharmaceutically acceptable salt thereof.

16. The compound of claim 11, wherein V is 3-chlorophenyl,
and B 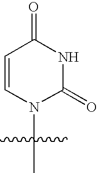
is or a pharmaceutically acceptable salt thereof.

17. The compound of claim 11, wherein said compound has the formula: 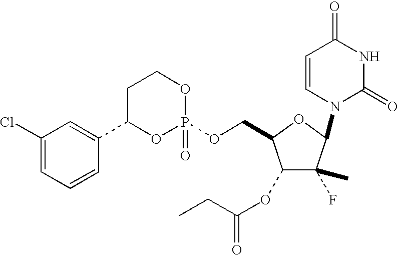
or a pharmaceutically acceptable salt thereof

18. A composition comprising a compound according to claim 11 or a pharmaceutically acceptable salt thereof and a pharmaceutically acceptable carrier, diluent, stabilizer or excipient.

19. The composition of claim 18, wherein said compound is

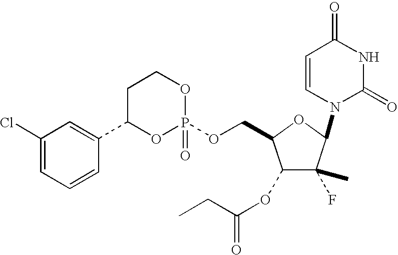

20. A method of treating hepatitis C infection or viral hepatitis comprising the administration of a compound or a pharmaceutically acceptable salt thereof according to claim 11 to an individual infected with hepatitis C or having viral hepatitis.

21. The method of claim 20, wherein said compound is [chemical structure]
